(19) United States
(12) Patent Application Publication
(10) Pub. No.: US 2023/0097440 A1
(43) Pub. Date: Mar. 30, 2023

(54) METHODS FOR MODULATION OF LIPOPROTEIN LIPASE AND APOLIPOPROTEIN C2 EXPRESSION AND/OR ACTIVITY IN THE TREATMENT OF PERIPHERAL AND CENTRAL NERVOUS SYSTEM TISSUE DISEASE STATES

(71) Applicant: ENDECE LLC, Mequon, WI (US)

(72) Inventor: James G. Yarger, Cedarburg, WI (US)

(21) Appl. No.: 17/970,293

(22) Filed: Oct. 20, 2022

Related U.S. Application Data
(62) Division of application No. 16/936,705, filed on Jul. 23, 2020, now abandoned.
(60) Provisional application No. 62/877,614, filed on Jul. 23, 2019.

(51) Int. Cl. C07J 1/00; A61K 9/00; A61P 37/00

(52) U.S. Cl. CPC C07J 1/0066; A61K 9/0073; A61P 37/00; A61K 9/0043; A61K 9/0078

(57) ABSTRACT

Methods for modulating lipoprotein lipase (LPL) and Apoliprotein C2 (ApoC2) expression and/or activity in the treatment of peripheral and central nervous system tissue disease states with C-6 substituted estradiol derivatives are presented herein.

### CROSS-REFERENCE TO RELATED APPLICATIONS

The present application claims priority to U.S. Provisional Patent Application No. 62/877,614 that was filed 23 Jul. 2019, the entire contents of which are hereby incorporated by reference.

### BACKGROUND OF THE INVENTION

Lipoprotein lipase (LPL) and apolipoprotein C2 (ApoC2) are key enzymes in lipid metabolism and involved in the hydrolysis of triglyceride (TG)-rich lipoproteins. There is growing body of literature to suggest that one or both of these proteins are important in the central nervous system (CNS). However, it remains unresolved as to whether their expression or activity can be used therapeutically for treating peripheral tissue disease states. Likewise, there remains an on-going concern in the art to identify compounds useful in the modulation of LPL and ApoC2 expression and/or activity and in conjunction with one or more such therapeutic treatments.

### BRIEF SUMMARY OF THE INVENTION

In light of the foregoing, it can be an object of the present invention to provide one or more methods of modulating LPL or ApoC2 expression and/or activity in the treatment of various peripheral and central nervous system (CNS) tissue disease states, as well as one or more compounds useful in conjunction therewith. It will be understood by those skilled in the art that one or more aspects of this invention can meet certain objectives, while one or more other aspects can meet certain other objectives. Each objective may not apply equally, in all its respects, to every aspect of this invention. As such, the following objects can be viewed in the alternative with respect to any one aspect of this invention.

It can also be an object of the present invention to provide one or more methods to up-regulate expression, abundance and/or activity of LPL and/or ApoC2 in the treatment of one or more such peripheral or CNS tissue disease states.

It can also be an object of the present invention to provide one or more methods of utilizing such up-regulation to switch, polarize or otherwise alter microglia or macrophages from a pro-inflammatory phenotype to an anti-inflammatory phenotype.

It can also be an object of the present invention to provide one or more methods of utilizing such phenotype modulation to treat inflammation in peripheral or CNS tissue.

It can also be an object of the present invention to provide one or more methods of utilizing such microglial or macrophage polarization or alteration, in adipose tissue, in the treatment and/or prevention of type-2 diabetes.

It can also be an object of the present invention to provide one or more methods of utilizing such up-regulation to enhance, affect or otherwise modulate lipid metabolism.

It can also be an object of the present invention to provide one or more methods of modulating lipid metabolism in the treatment and/or prevention of cardiovascular disease and obesity.

It can be another object of the present invention, alone or in conjunction with one or more of the preceding objectives, to provide one or more compounds useful in conjunction with such up-regulation, microglial or macrophage polarization and/or lipid metabolism, in the treatment and/or prevention of various peripheral tissue disease states, including but not limited to type-2 diabetes, cardiovascular disease and obesity, or CNS tissue disease states, including but not limited to multiple sclerosis, Alzheimer's disease, or Niemann-pick type C, neuromylelitis optica spectrum disorder.

Other objectives, features, benefits and advantages of the present invention will be apparent from this summary and descriptions of certain embodiments, and will be readily apparent to those skilled in the art having knowledge of the treatment of such disease states and compounds useful in conjunction therewith. Such objects, features, benefits and advantages will be apparent from the above as taken into conjunction with the accompanying examples, data, figures and all these low inferences to be drawn therefrom, together with consideration of the references incorporated herein.

In part, the present invention can be directed to a method for modulating at least one of LPL or ApoC2 expression and activity or, alternatively, treating a peripheral or central nervous system tissue disease state including but not limited to inflammation, including inflammation associated with an infection, type-2 diabetes, cardiovascular disease and obesity. Such a method can comprise (i) providing a compound of structural formula I.

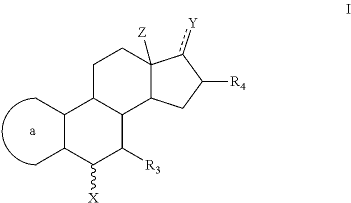

wherein the “a” ring is selected from the group consisting of

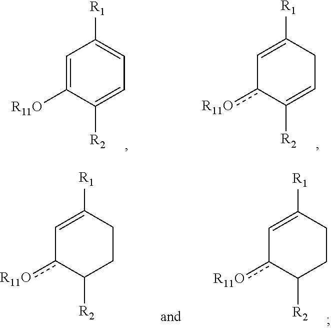

R₁, R₂, R₃ and R₄ are independently selected from hydrogen, C₁-C₆ alkyl, halo, a sulfate, a glucuronide, —OH, a bulky group, aryl, cycloalkyl, heteroaryl, heterocycloalkyl, —NH(CH₂)ₙCH₃; a phosphate group, and a phosphinate group; Rn is selected from H, C₁-C₆ alkyl, halogen, a sulfate, a glucoronide, —SO₂NH₂, —COOH, —CN, —CH₂CN—, —NHCN—, —CHO, ═CHOCH₃, —COO salt, —OSO₂alkyl, —NH₂, and —NHCO(CH₂)ₙ; X is selected from C₁-C₁₂ alkyl, C₂-C₁₂ alkenyl, C₂-C₁₂ alkynyl, halogen, a glucoronide, —NH₂, —SO₂NH₂, —COOH, —CN, —CH₂CN, —NHCN, —CHO, —COOsalt, —OSO₂alkyl, —SH, —SCH₃, —CH[(CH₂)ₙCH₃]COOCH₃, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—NH—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-NH—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-NH—(CH₂)ₙCH₃, —(CH₂)ₘ—OH, —(CH₂)ₘ—NH₂, —(CH₂)ₘ—O—NH₂, —(CH₂)ₘ—S—NH₂, —NH(CH₂)ₘCH₃, —NH(CH₂)ₘOCH₃, —NH(CH₂)ₘCHOH—COOH, —N(CH₃)₂, —(CH₂)ₘ(NH)CH₂OH, —NHCOOH, —(CH₂)ₘNHCOOH, —NO₂, —SCN, —SO₂alkyl, —B(OH)₂, —(CH₂)ₘN(CH₃)—SO₂—NH₃, —(CH₂)ₘ—NH—SO₂—NH₂, —NHC(═S)CH₃, and —NHNH₂; Y is selected from hydrogen, ═O, —OCO(C₁-C₂₀ alkyl) and —OH; and Z is H or methyl; wherein m is an integer between 0-20, n is an integer between 0-8, the [symbol] symbol represents either a single or a double bond capable of forming a keto group at position 3 and/or 17; and the [symbol] symbol represents any type of bond regardless of the stereochemistry; and the respective enantiomers, other stereochemical isomers, hydrates, solvates, tautomers and pharmaceutically acceptable salts of said compounds; and (ii) administering an effective amount of such a compound to a subject in need thereof.

Such a method can comprise enhancing or up-regulating LPL expression and/or activity, such enhancement or up-regulation as can provide a microglial or macrophage anti-inflammatory phenotype and/or modulate lipid metabolism. Regardless, in certain non-limiting embodiments, one or more such compounds can be provided as a composition comprising a pharmaceutically-acceptable carrier.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1F show NDC-1308 increases LPL expression and activity in neonatal primary rat microglia. Neonatal primary rat microglia (N=3 per group) were treated with increasing concentrations of compound NDC-1308 for 72 hours prior to analysis. FIG. 1A shows relative gene expression of Lipoprotein Lipase (LPL). FIG. 1B shows relative gene expression of Inducible Nitric Oxide synthase (iNOS). FIG. 1C shows relative gene expression of Arginase 1 (Arg1). FIG. 1D shows relative gene expression of Transmembrane protein 110 (Tmem119). FIG. 1E shows LPL was released from the cell surface (N=5) with a heparin containing buffer and was incubated with a synthetic triglyceride-containing lipoprotein to determine LPL hydrolytic activity. FIG. 1F shows protein expression from (N=3, pooled) microglia treated with NDC-1308.

FIGS. 2A-2C show compound NDC-1308 increased LPL expression, and can improve remyelination following lysolecithin mediated demyelination. FIG. 2A shows cerebellar slices were maintained in culture for 7 days, NDC-1308 was then added for 72 hours (optimized timescale from primary microglial cell work). Slices were demyelinated with lysolecithin and allowed to remyelinate for 7 days. Slices were stained with Allograft inflammatory factor 1 (Iba1), proteolipid protein (PLP1) and Neurofilament (NF). FIG. 2B shows percent myelination was determined by analyzing NF/PLP. FIG. 2C shows relative expression of LPL in ex vivo cerebellar slices that were treated with NDC-1308 (1 uM) for 72 hours prior to lysolecithin-mediated demyelination. Slices were allowed to recover for 3 days (with respective drug treatments) prior to analysis.

### DETAILED DESCRIPTION OF THE INVENTION

Unless defined otherwise, all technical and scientific terms used herein have the same meaning as is commonly understood by one of skill in the art to which this invention belongs and shall be understood to have the meanings described below. All publications and patents referred to herein are incorporated by reference in their entirety. Unless otherwise specified, a reference to a particular compound includes all such isomeric forms, including racemic and other mixtures thereof. Unless otherwise specified, a reference to a particular compound also includes ionic, salt, solvate (e.g., hydrate), protected forms, prodrugs, and other stereoisomers thereof, for example, as discussed herein.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19, and discussed herein.

The term “treatment,” or “therapy” as used herein in the context of treating a condition, pertains generally to treatment and therapy of a mammalian subject, whether of a human or a non-human animal (e.g., in veterinary applications), in which some desired therapeutic effect is achieved, for example, the inhibition of the progress of the condition, and includes a reduction in the rate of progress, a halt in the rate of progress, amelioration of the condition, and/or cure of the condition. Treatment as a prophylactic measure is also included. Treatment includes combination treatments and therapies, in which two or more treatments or therapies are combined, for example, sequentially or simultaneously. Examples of treatments and therapies include, but are not limited to, chemotherapy (the administration of active agents, including, e.g., drugs, antibodies (e.g., as in immunotherapy), anti-inflammatory, prodrugs (e.g., employing protecting groups including phosphoric acid derivatives and phosphinates at suitable positions such as position 3 or 17, other compounds used for photodynamic therapy, GDEPT, ADEPT, etc.), surgery, radiation therapy, and gene therapy. Preferable combination treatments include the methods of the invention in combination with existing therapies.

The term “stereochemical isomer” as used herein, refers to isomers that differ from each other only in the way the atoms are oriented in space. The two stereoisomers particularly of importance in the instant invention are enantiomers and diastereomers depending on whether or not the two isomers are mirror images of each other. In the preferred embodiment, the claimed formulations comprise such compounds that isolated, resolved and are “substantially free of other isomers.”

The term “therapeutically-effective amount,” as used herein, pertains to that amount of an active compound, or a material, composition or dosage form comprising an active compound, which is effective for producing some desired therapeutic effect, commensurate with a reasonable benefit/risk ratio. By the term “effective amount” is meant an amount that can bring about a detectable effect, generally.

The term “subject” may be interchangeable with “patient” or “individual” and means an animal, which may be a human or non-human animal, in need of treatment. A “subject in need of treatment” may include a subject having a disease, disorder, or condition that is responsive to therapy with the compounds disclosed here herein. For example, a “subject in need of treatment” may include a subject having a peripheral or central nervous system disease state that may be treated by modulating LPL and/or ApoC2 expression or activity and/or switching, polarizing, or altering an inflammatory phenotype. In some embodiments, the subject in need of a treatment may include a subject having a disease, disorder, or condition associated with lipid metabolism and/or inflammation. In some embodiments, the subject may be in need of a treatment for a type-2 diabetes, cardiovascular disease, or obesity. In some embodiments, the subject may be in need of a treatment for multiple sclerosis, Alzheimer's disease, or Niemann-pick type C, neuromylelitis optica spectrum disorder. In some embodiments, the subject may be in need of a treatment for an infection, including, without limitation, viral infections associated with inflammation of the respiratory or pulmonary system.

Inflammation of the peripheral nervous system may be associated with an infection, such as a viral infection of the pulmonary or respiratory system. The present technology is useful for the treatment of subjects suffering from pulmonary viral infections, including COVID-19 or seasonal influenza, and acute respiratory distress syndrome (ARDS) or severe acute respiratory syndrome (SARS) related viral diseases. Respiratory distress and failure is influenced by M1, pro-inflammatory macrophages. When the pro-inflammatory state gets out of control, respiratory failure occurs along with additional problems associated with cytokine over expression. By polarizing the macrophages to the M2, anti-inflammatory state, the inflammation may be reduced within the respiratory or pulmonary tissues. The present technology provides for switching, polarizing, or altering an inflammatory phenotype. As a result, the present technology is capable of modulating inflammatory and reparative gene expression, down-regulating the former or upregulating the latter.

Inflammation is a fundamental immune response designed to protect the body, tissues, and cells, from harm arising from both endogenous and exogenous sources. Microglia and macrophages use an array of immune receptors, such as toll-like receptors (TLRs), nucleotide-binding oligomerization domains (NODs), NOD-like receptors, scavenger receptors, and the like to recognize harmful stimuli to produce inflammatory cytokines by upregulating inflammatory genes, such as TNF-α, IL-6, IL-12, IL-23, IL-1β, IFNγ, nitrogen monoxide via iNOS induction, chemokines, and the like. This cytokine production is essential for polarization of microglia and macrophages into a pro-inflammatory or M1 phenotype. In many cases, this response is protective and down-regulated once the damage or pathogen has been mitigated, but unregulated, long-term or chronic inflammation can lead to tissue destruction and/or disease states.

In contrast to pro-inflammatory phenotypes, microglia and macrophages can also possess an anti-inflammatory or M2 phenotype. Microglia and macrophages having an anti-inflammatory phenotype express cytokines and receptors by upregulating reparative genes that are implicated in inhibiting inflammation and restoring homeostasis and down-regulating inflammatory genes. Anti-inflammatory cytokines, such as TGF-β, IL-4, IL-10, and IL-13, growth factors such as vascular endothelial growth factor (VEGF), brain-derived neurotrophic factor (BDNF), platelet-derived growth factor (PDGF), may be some of the reparative genes upregulated.

One of the best characterized markers for identifying an anti-inflammatory phenotype is the enzyme arginase 1 (Arg1), which converts arginine to polyamines, proline, and ornithine that contribute to wound healing and matrix deposition. By using arginine, which is the same substrate used by inducible nitric oxide synthase (iNOS), Arg1 can effectively outcompete iNOS to downregulate production of nitric oxide. Thus, iNOS and Arg1 represent a relatively straightforward set of markers to follow M1 versus M2 phenotypes. Other markers used for identifying M2 cells include Ym1, a heparin-binding lectin, FIZZ1, which promotes deposition of extracellular matrix, and CD206, a mannose receptor, CD163, which binds and internalizes or phagocytoses mannosylated ligands, and TREM2, which is involved with debris clearance.

The present technology is useful for the treatment of subjects suffering inflammation within CNS tissues that may result in demyelination by inducing remyelination to restore function. In some instances, the subject suffers from Alzheimer's disease, multiple sclerosis, Niemann-pick type C, neuromylelitis optica spectrum disorder, or the like. CNS tissue inflammation is influenced by M1, pro-inflammatory microglial cells. When the pro-inflammatory state gets out of control, demyelination occurs along with additional problems associated with cytokine over expression. Polarizing the microglial cells to the M2, anti-inflammatory state, the inflammation may be reduced within the CNS tissues, resulting in remyelination. In some embodiments, the subject may receive direct administration of the compound directly into the pulmonary or respiratory system via delivery by inhaler, nebulizer, or nasal spray device.

The term “tissue” refers generally to specialized cells which may perform a particular function. The term “tissue” may refer to an individual cell or a plurality or aggregate of cells, for example, membranes, blood or organs. Tissue may be peripheral or CNS tissue. The term “tissue” also includes reference to an abnormal cell or a plurality of abnormal cells. Exemplary tissues include respiratory or pulmonary tissue, including cells of the lungs, bronchi, trachea, larynx, nose, or sinus, breast tissue, including breast cells, membranous tissues, including endothelium and epithelium, laminae, connective tissue, including interstitial tissue, and tumors.

By “alkyl” in the present invention is meant a straight or branched chain alkyl radical having 1-20, and preferably from 1-12, carbon atoms. Examples include but are not limited to methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. Each alkyl group may be optionally substituted with one, two or three substituents such as, for example, a halo, cycloalkyl, aryl, alkenyl or alkoxy group and the like.

By “aryl” is meant an aromatic carbocylic radical having a single ring (e.g. phenyl), multiple rings (e.g. biphenyl) or multiple fused rings in which at least one is aromatic (e.g. 1,2,3,4-tetrahydronaphthyl). The aryl group can also be optionally mono-, di-, or trisubstituted with, for example, halo, alkyl, alkenyl, cycloalkyl or alkoxy and the like.

By “heteroaryl” is meant one or multiple fused aromatic ring systems of 5-, 6- or 7-membered rings containing at least one and up to four heteroatoms selected from nitrogen, oxygen or sulfur. Examples include but are not limited to furanyl, thienyl, pyridinyl, pyrimidinyl, benzimidazolyl and benzoxazolyl. The heteroaryl group can also be optionally mono-, di-, or trisubstituted with, for example, halo, alkyl, alkenyl, cycloalkyl or alkoxy and the like.

By “cycloalkyl” is meant a carbocylic radical having a single ring (e.g. cyclohexyl), multiple rings (e.g. bicyclohexyl) or multiple fused rings (e.g. bicyclo[2.2.1]heptane). The cycloalkyl group can optionally contain from 1 to 4 heteroatoms. In addition, the cycloalkyl group may have one or more double bonds. The cycloalkyl group can also be optionally mono-, di-, or trisubstituted with, for example, halo, alkyl, alkenyl, aryl or alkoxy and the like.

By “alkoxy” is meant an oxy-containing radical having an alkyl portion. Examples include, but are not limited to, methoxy, ethoxy, propoxy, butoxy and tert-butoxy. The alkoxy group can also be optionally mono-, di-, or trisubstituted with, for example, halo, aryl, cycloalkyl or alkoxy and the like.

By “alkenyl” is meant a straight or branched hydrocarbon radical having from 2 to 20, and preferably from 2-6, carbon atoms and from one to three double bonds and includes, for example, ethenyl, propenyl, 1-but-3-enyl, 1-pent-3-enyl, 1-hex-5-enyl. The alkenyl group can also be optionally mono-, di-, or trisubstituted with, for example, halo, aryl, cycloalkyl or alkoxy and the like.

“Halo” or “halogen” is a halogen radical of fluorine, chlorine, bromine or iodine.

By “glucuronide” is meant a glycoside radical of glucuronic acid.

The term “sulfate” refers to a radical having the general formula —OS(O)₂—OR′, wherein R′ is hydrogen, a metal or an alkyl group.

The term “phosphate” refers to a radical having the general formula —OP(O)(OR′)₂, wherein each R′ is independently hydrogen, a metal or an alkyl group.

The term “phosphinate” refers to a radical having the general formula —OP(O)(R′)₂, wherein each R′ is independently hydrogen, a metal or an alkyl group.

By “bulky group” is meant a substituent that produces steric hindrance about the space to which it is attached, e.g. a t-butyl group.

The term “amino alkyl” as used herein refers to an alkyl group with an amino group on it, for example, H₂N—CH₂—, H₂N—CH₂CH₂—, Me₂NCH₂—, etc., wherein the point of attachment is a carbon of the alkyl chain; and the term “alkyl amino” as used herein refers to an amino group with an alkyl group attached to the nitrogen atom, for example, CH₃NH—, EtNH—, iPr-NH—, etc., wherein the point of attachment is via the nitrogen atom of the amino group. All other terms wherein successive radicals are employed will adhere to a similar rule.

In an embodiment of the present invention, compounds useful in conjunction with methods of this invention have the general structure shown in Formula (Ia) below:

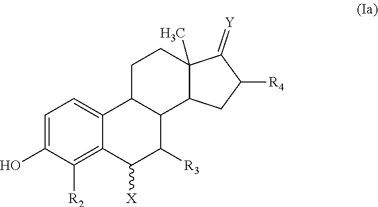

wherein R₂, R₃, R₄, X and Y are as defined above for Formula (I). Even more preferably, Y is selected from ═O and —OH; R₄ is selected from hydrogen, halo and C₁-C₆ alkyl; R₂ is selected from hydrogen, —OH and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, —NH(CH₂)ₘOCH₃, —NH(CH₂)ₘCHOH—COOH, —(CH₂)ₘ(NH)CH₂OH, —(CH₂)ₘNHCOOH, —(CH₂)ₘN(CH₃)—SO₂—NH₃, and —(CH₂)ₘ—NH—SO₂—NH₂; m is an integer from 1-20; n is an integer from 0-8; and the [symbol] symbol represents either a single or a double bond. Yet even more preferably, Y is (S)—OH; R₄ is selected from hydrogen or alkyl; R₂ is hydrogen; R₃ is hydrogen; and X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-12; n is an integer from 0-4; and the C-13 methyl is in the (S) configuration.

Yet another embodiment of the present invention is directed to methods using compounds of a Formula (Ib):

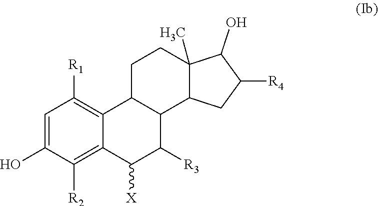

wherein R₁ R₂, R₃, R₄ and X are as defined above for Formula (I). Even more preferably, R₁ is selected from hydrogen, —OH and halo; R₄ is selected from hydrogen, halo and C₁-C₆ alkyl; R₂ is selected from hydrogen and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘN(CH₃)—SO₂—NH₃, and —(CH₂)ₘ—NH—SO₂—NH₂; m is an integer from 1-20; and n is an integer from 0-8. Yet even more preferably, R₁ is hydrogen; R₄ is selected from hydrogen or alkyl; R₂ is hydrogen; R₃ is hydrogen; and X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-12; n is an integer from 0-4; and both the C-13 methyl and C-17 hydroxyl are in the (S) configuration.

Still another embodiment of the invention is directed to methods using a compound of a Formula (Ic):

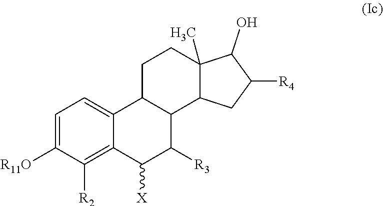

wherein R₁₁, R₂, R₃, R₄ and X are as defined above for Formula (I). Even more preferably, R₁₁ is hydrogen or C₁-C₆ alkyl; R₄ is selected from hydrogen, halo and C₁-C₆ alkyl; R₂ is selected from hydrogen and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S-CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘN(CH₃)—SO₂—NH₃, and —(CH₂)ₘ—NH—SO₂—NH₂; m is an integer from 1-20; and n is an integer from 0-8. Yet even more preferably, R₁₁ is hydrogen; R₄ is selected from hydrogen or alkyl; R₂ is hydrogen; R₃ is hydrogen; and X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-12; n is an integer from 0-4; and both the C-13 methyl and C-17 hydroxyl are in the (S) configuration.

Yet another embodiment of the present invention is directed to methods using a compound of a Formula (Id):

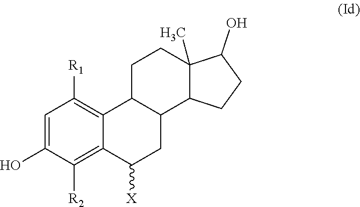

wherein R₁, R₂, and X are as defined above for Formula (I). Even more preferably, R₁ is selected from hydrogen, —OH and halo; R₂ is selected from hydrogen and halo; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘN(CH₃)—SO₂—NH₃, and —(CH₂)ₘ—NH—SO₂—NH₂; X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-20; and n is an integer from 0-8. Still even more preferably, R₁ and R₂ are hydrogen; m is an integer from 1-12; n is an integer from 0-4; and both the C-13 methyl and C-17 hydroxyl are in the (S) configuration.

Yet another embodiment of the present invention is directed to methods using a compound of a Formula (Ie):

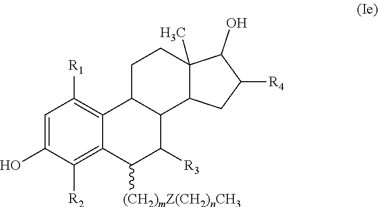

wherein m, n, R₁, R₂, R₃ and R₄ are as defined above for Formula (I), and Z is selected from —O—, —S— and —NH—. Preferably, m is 1-12, n is 0-4, R₁ is selected from hydrogen, —OH and halo; R₄ is selected from hydrogen, halo and C₁-C₆ alkyl; R₂ is selected from hydrogen and halo; R₃ is selected from hydrogen, halo and —OH; Z is selected from —O— and —S—; and both the C-13 methyl and C-17 hydroxyl are in the (S) configuration. More preferably, Z is —O—, R₁-R₄ are hydrogen, m is 4-8, n is 0-4, and optionally, the C-13 methyl and C-17-hydroxyl are (S)-configured and the C-6 substituent is (R)-configured.

Still another embodiment of the present invention is directed to methods using a compound of a Formula (If):

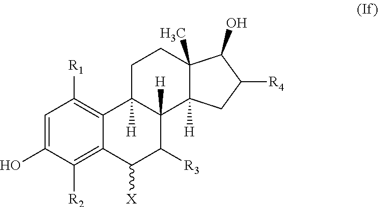

wherein R₁, R₂, R₃, R₄ and X are as defined above for Formula (I). Preferably, R₁ is selected from hydrogen, —OH and halo; R₄ is selected from hydrogen, halo and C₁-C₆ alkyl; R₂ is selected from hydrogen and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘN(CH₃)—SO₂—NH₃, and —(CH₂)ₘ—NH—SO₂—NH₂; X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-20; and n is an integer from 0-8. Alternatively, R₁, R₂, R₃ and R₄ are hydrogen; m is an integer from 1-12; and n is an integer from 0-4. More preferably, Z is —O—, R₁-R₄ are hydrogen, m is 4-8, n is 0-4, the C-13 methyl and C-17-hydroxyl are (S)-configured and the C-6 substituent is (R)-configured.

Still another embodiment of the present invention is directed to methods using a compound of a Formula (Ig):

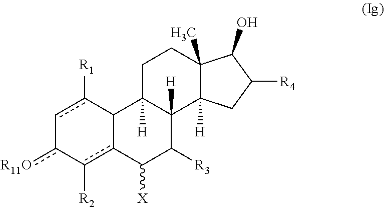

wherein R₁, R₂, R₃, R₄, R₁₁ and X are as defined above for Formula (I). Even more preferably, R₁ is selected from hydrogen, —OH and halo; R₄ is selected from hydrogen, halo and C₁-C₆ alkyl; R₂ is selected from hydrogen and halo; R₃ is selected from hydrogen, halo and —OH; and X is selected from C₁-C₁₂ alkyl, C₂-C₁₂ alkenyl, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—N—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-N—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-N—(CH₂)ₙCH₃, —(CH₂)ₘ—OH, —(CH₂)ₘ—O—NH₂, —(CH₂)ₘ—S—NH₂, —NH(CH₂)ₘCH₃, NH(CH₂)ₘOCH₃, —NH(CH₂)ₘCHOH—COOH, —(CH₂)ₘ(NH)CH₂OH, —(CH₂)ₘNHCOOH, —(CH₂)ₘN(CH₃)—SO₂—NH₃, and —(CH₂)ₘ—NH—SO₂—NH₂; X is selected from C₁-C₁₂ alkyl, C₂-C₁₂ alkenyl, —(CH₂)ₘ—O—CH₃, —(CH₂)ₘ—O—(CH₂)ₙCH₃, (CH₂)ₘ—S—CH₃, and —(CH₂)ₘ—S—(CH₂)ₙCH₃; m is an integer from 1-20; [symbol] OR₁₁ is either ═O or —OH; and n is an integer from 0-8. Still even more preferably, R₁, R₂, R₃ and R₄ are hydrogen; m is an integer from 1-12; and n is an integer from 0-4.

Specific examples of compounds of Formula (I) and (Ia)-(If) are shown below:

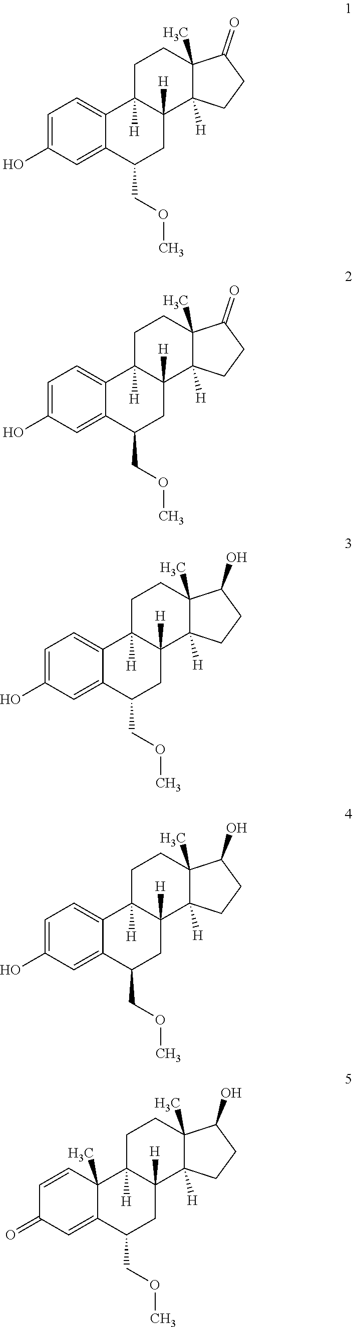

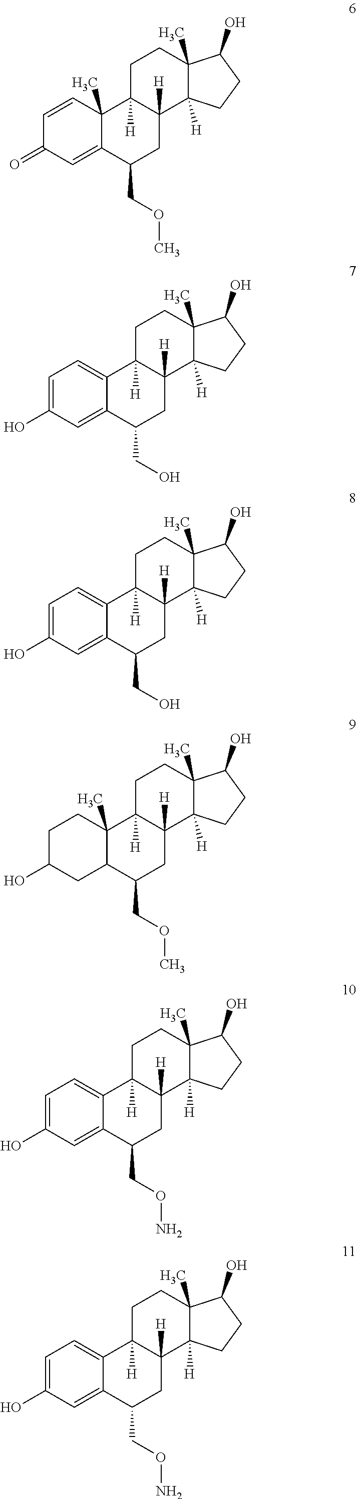

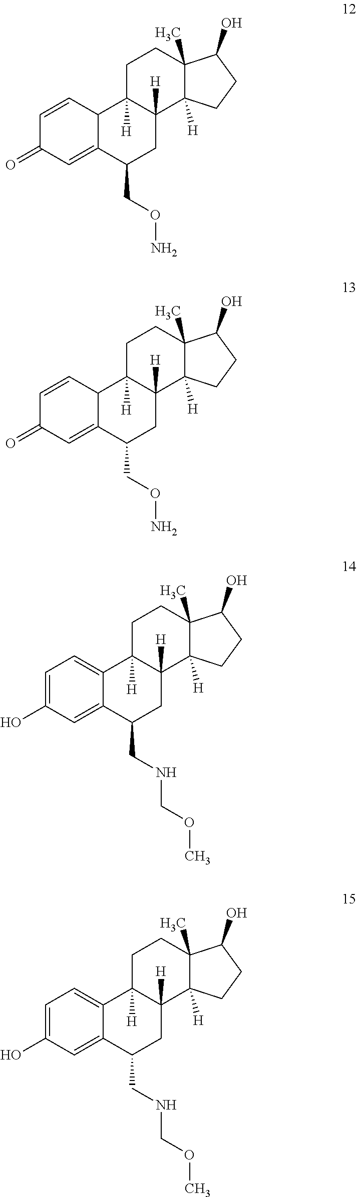

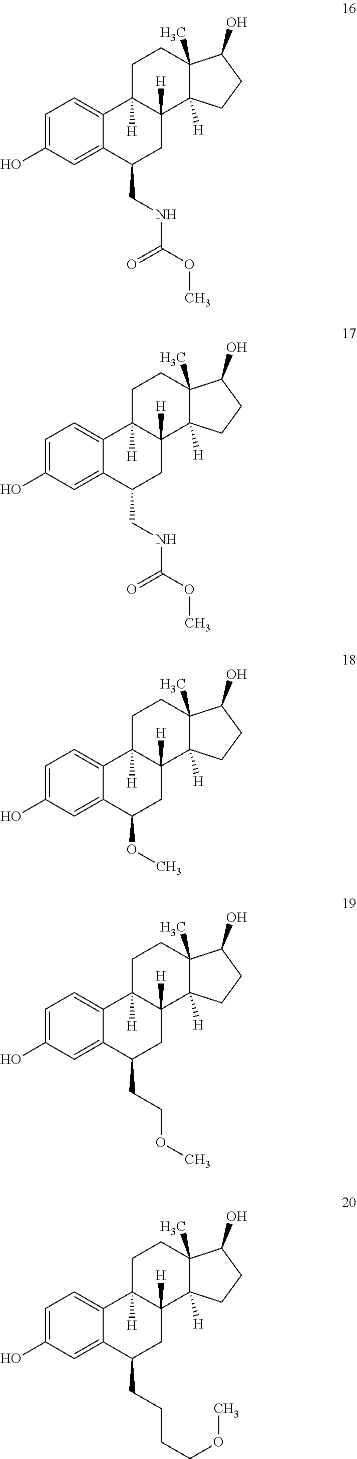

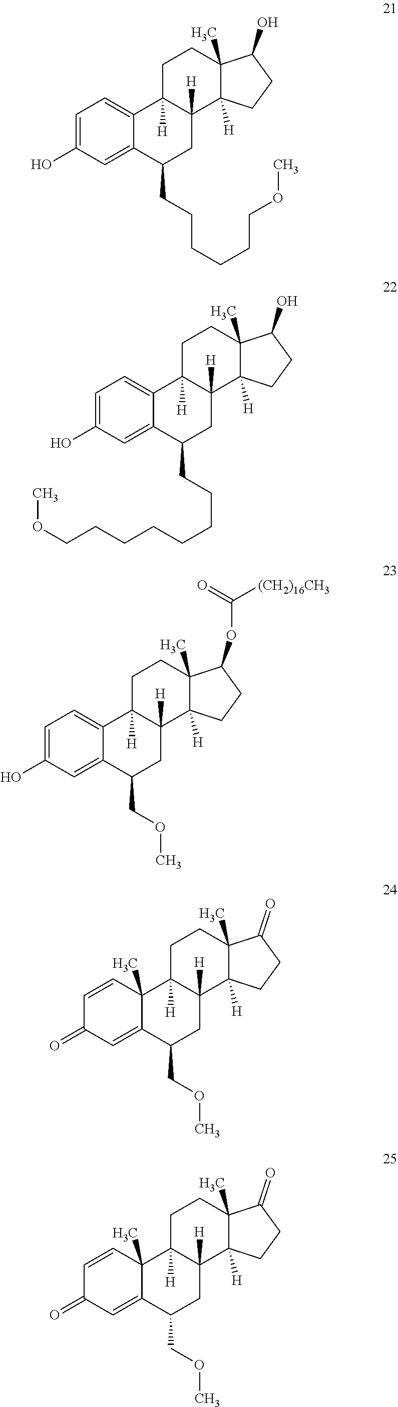

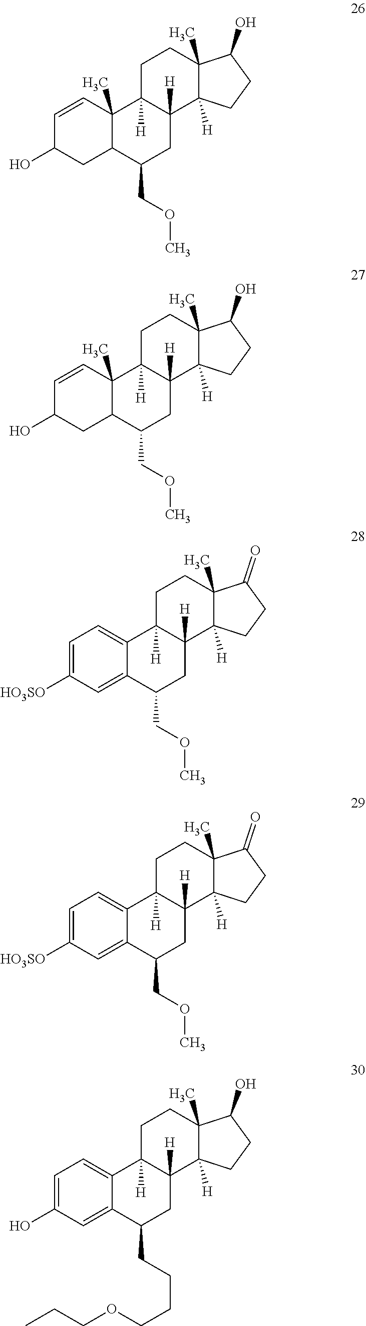

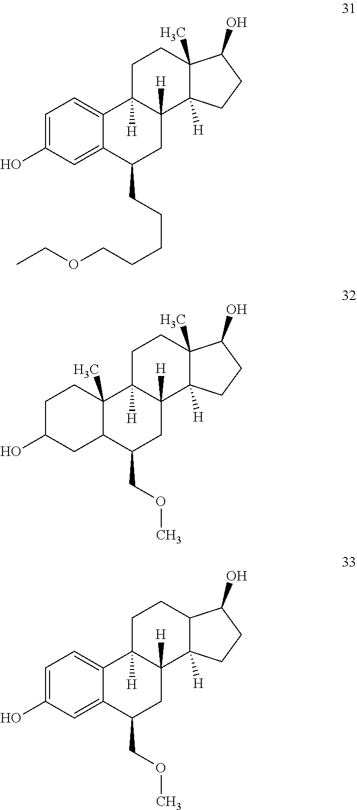

In certain non-limiting embodiments, compounds of the present invention can be used in conjunction or incorporated in a pharmaceutical composition. Such a composition can comprise one or more compounds selected from those discussed above, illustrated below or otherwise inferred herein, and combinations thereof. In certain embodiments, such a composition can comprise a pharmaceutically-acceptable excipient, carrier, or diluent component. Without limitation, such a composition can comprise a racemic mixture of compounds. In certain embodiments, such a compound can be present as the S and R enantiomer, preferably its isolated and purified form which is substantially free of the other isomer.

Pharmaceutical compositions comprising the compounds may be adapted for administration by any appropriate route, for example by the oral (including buccal or sublingual), rectal, nasal, topical (including buccal, sublingual or transdermal), vaginal, parenteral (including subcutaneous, intramuscular, intravenous or intradermal), or pulmonary route. Such formulations may be prepared by any method known in the art of pharmacy, for example by bringing into association the active ingredient with the carrier(s) or excipient(s). Examples of pharmaceutical compositions for oral administration include capsules, syrups, concentrates, powders and granules. Examples of pharmaceutical compositions for pulmonary administration include solutions, suspensions, and powders suitable for delivery via metered dose inhalers, dry powder inhalers, nebulizers, nasal spray device, and the like.

The compounds of the present invention may have asymmetric centers and may occur as a racemate, a racemic mixture or as individual and purified diastereomers or enantiomers such as (named via ChemDraw Ultra, Version 11.0(3) or 12.0) (6S,8R,9S,13S,14S)-3-hydroxy-6-(methoxymethyl)-13-methyl-7,8,9,11,12,13,15,16-octahydro-6H-cyclopenta[a]phenanthren-17(14H)-one (compound 1); (6R,8R,9S,13S,14S)-3-hydroxy-6-(methoxymethyl)-13-methyl-7,8,9,11,12,13,15,16-octahydro-6H-cyclopenta[a]phenanthren-17(14H)-one (compound 2); (6S,8R,9S,13S,14S)-6-(methoxymethyl)-13-methyl-7,8,9,11,12,13,14,15,16,17-decahydro-6H-cyclopenta[a]phenanthrene-3,17-diol (compound 3); (6R,8R,9S,13S,14S)-6-(methoxymethyl)-13-methyl-7,8,9,11,12,13,14,15,16,17-decahydro-6H-cyclopenta[a]phenanthrene-3,17-diol (compound 4); (6S,8R,9S,10R,13S,14S)-17-hydroxy-6-(methoxymethyl)-10,13-dimethyl-6,7,8,9,10,11,12,13,14,15,16,17-dodecahydro-3H-cyclopenta[a]phenanthren-3-one (compound 5); (6R,8R,9S,10R,13S,14S)-17-hydroxy-6-(methoxymethyl)-10,13-dimethyl-6,7,8,9,10,11,12,13,14,15,16,17-dodecahydro-3H-cyclopenta[a]phenanthren-3-one (compound 6); (6S,8R,9S,13S,14S)-6-(hydroxymethyl)-13-methyl-7,8,9,11,12,13,14,15,16,17-decahydro-6H-cyclopenta[a]phenanthrene-3,17-diol (compound 7); (6R,8R,9S,13S,14S)-6-(hydroxymethyl)-13-methyl-7,8,9,11,12,13,14,15,16,17-decahydro-6H-cyclopenta[a]phenanthrene-3,17-diol (compound 8); (6R,8R,9S,10R,13S,14S)-6-(methoxymethyl)-10,13-dimethylhexadecahydro-1H-cyclopenta[a]phenanthrene-3,17-diol (compound 9); (6R,8R,9S,13S,14S)-6-((aminooxy)methyl)-13-methyl-7,8,9,11,12,13,14,15,16,17-decahydro-6H-cyclopenta[a]phenanthrene-3,17-diol (compound 10); (6S,8R,9S,13S,14S)-6-((aminooxy)methyl)-13-methyl-7,8,9,11,12,13,14,15,16,17-decahydro-6H-cyclopenta[a]phenanthrene-3,17-diol (compound 11); (6R,8R,9S,13S,14S)-6-((aminooxy)methyl)-17-hydroxy-13-methyl-6,7,8,9,10,11,12,13,14,15,16,17-dodecahydro-3H-cyclopenta[a]phenanthren-3-one (compound 12); (6S,8R,9S,13S,14S)-6-((aminooxy)methyl)-17-hydroxy-13-methyl-6,7,8,9,10,11,12,13,14,15,16,17-dodecahydro-3H-cyclopenta[a]phenanthren-3-one (compound 13); (6R,8R,9S,13S,14S)-6-(((methoxymethyl)amino)methyl)-13-methyl-7,8,9,11,12,13,14,15,16,17-decahydro-6H-cyclopenta[a]phenanthrene-3,17-diol (compound 14); (6S,8R,9S,13S,14S)-6-(((methoxymethyl)amino)methyl)-13-methyl-7,8,9,11,12,13,14,15,16,17-decahydro-6H-cyclopenta[a]phenanthrene-3,17-diol (compound 15); 1-((((6R,8R,9S,13S,14S)-3,17-dihydroxy-13-methyl-7,8,9,11,12,13,14,15,16,17-decahydro-6H-cyclopenta[a]phenanthren-6-yl)methyl)amino)propan-2-one (compound 16); 1-((((6S,8R,9S,13S,14S)-3,17-dihydroxy-13-methyl-7,8,9,11,12,13,14,15,16,17-decahydro-6H-cyclopenta[a]phenanthren-6-yl)methyl)amino)propan-2-one (compound 17); (6R,8R,9S,13S,14S)-6-methoxy-13-methyl-7,8,9,11,12,13,14,15,16,17-decahydro-6H-cyclopenta[a]phenanthrene-3,17-diol (compound 18); (6S,8R,9S,13S,14S)-6-(2-methoxyethyl)-13-methyl-7,8,9,11,12,13,14,15,16,17-decahydro-6H-cyclopenta[a]phenanthrene-3,17-diol (compound 19); (6R,8R,9S,13S,14S)-6-(4-methoxybutyl)-13-methyl-7,8,9,11,12,13,14,15,16,17-decahydro-6H-cyclopenta[a]phenanthrene-3,17-diol (compound 20); (6R,8R,9S,13S,14S)-6-(6-methoxyhexyl)-13-methyl-7,8,9,11,12,13,14,15,16,17-decahydro-6H-cyclopenta[a]phenanthrene-3,17-diol (compound 21); (6R,8R,9S,13S,14S)-6-(6-methoxyoctyl)-13-methyl-7,8,9,11,12,13,14,15,16,17-decahydro-6H-cyclopenta[a]phenanthrene-3,17-diol (compound 22); (6R,8R,9S,13S,14S)-3-hydroxy-6-(methoxymethyl)-13-methyl-7,8,9,11,12,13,14,15,16,17-decahydro-6H-cyclopenta[a]phenanthren-17-yl stearate (compound 23); (6R,8R,9S,10R,13S,14S)-6-(methoxymethyl)-10,13-dimethyl-7,8,9,10,11,12,13,14,15,16-decahydro-3H-cyclopenta[a]phenanthrene-3,17(6H)-dione (compound 24); (6S,8R,9S,10R,13S,14S)-6-(methoxymethyl)-10,13-dimethyl-7,8,9,10,11,12,13,14,15,16-decahydro-3H-cyclopenta[a]phenanthrene-3,17(6H)-dione (compound 25); (6R,8R,9S,10R,13S,14S)-6-(methoxymethyl)-10,13-dimethyl-4,5,6,7,8,9,10,11,12,13,14,15,16,17-tetradecahydro-3H-cyclopenta[a]phenanthrene-3,17-diol (compound 26); (6S,8R,9S,10R,13S,14S)-6-(methoxymethyl)-10,13-dimethyl-4,5,6,7,8,9,10,11,12,13,14,15,16,17-tetradecahydro-3H-cyclopenta[a]phenanthrene-3,17-diol (compound 27); (6S,8R,9S,13S,14S)-6-(methoxymethyl)-13-methyl-17-oxo-7,8,9,11,12,13,14,15,16,17-decahydro-6H-cyclopenta[a]phenanthren-3-yl hydrogen sulfate (compound 28); (6R,8R,9S,13S,14S)-6-(methoxymethyl)-13-methyl-17-oxo-7,8,9,11,12,13,14,15,16,17-decahydro-6H-cyclopenta[a]phenanthren-3-yl hydrogen sulfate (compound 29); (6R,8R,9S,13S,14S)-13-methyl-6-(4-propoxybutyl)-7,8,9,11,12,13,14,15,16,17-decahydro-6H-cyclopenta[a]phenanthrene-3,17-diol (compound 30); (6R,8R,9S,13S,14S)-13-methyl-6-(5-ethoxypentyl)-7,8,9,11,12,13,14,15,16,17-decahydro-6H-cyclopenta[a]phenanthrene-3,17-diol (compound 31); (6R,8R,9S,10R,13S,14S)-6-(methoxymethyl)-10,13-dimethylhexadecahydro-1H-cyclopenta[a]phenanthrene-3,17-diol (compound 32); and (6R,8S,9S,14S,17S)-6-(methoxymethyl)-7,8,9,11,12,13,14,15,16,17-decahydro-6H-cyclopenta[a]phenanthrene-3,17-diol (compound 33).

The compounds of the methods of the invention are prepared as described in U.S. Pat. No. 10,174,070, incorporated herein by reference, and pertain to a method for preparing a 6-hydroxymethyl, 6-alkoxyalkyl, 6-alkylthioalkyl, 6-aminomethoxy, 6-methylaminomethoxy, or 6-methoxyamine derivatives of estradiol. Reaction schemes for preparing estradiol derivatives is given below, Schemes 1-3. Such methods can comprise reaction of a t-butyldimethylsilyl derivative of estradiol with LIDAKOR/THF/formaldehyde to obtain a 6-hydroxylated compound followed by such steps as: (i) hydrolysis to obtain 6-hydroxymethyl derivative of estradiol; and/or (ii) treatment with dimethylsulfate followed by hydrolysis to obtain 6-methyloxymethyl derivative of estradiol. Compound 1 can be obtained by further oxidation of compound 3 at the C-17 hydroxyl position. Compound 33 and other dimethyl compounds can be prepared according to United States Pub. No. 2012/0071455 (incorporated herein by reference).

In an alternative approach, the compounds of the present invention can also be prepared by a method comprising such steps as: (i) protecting an estrodial compound, (ii) acylating the protected estradiol compound at the benzylic 6-position with LIDAKOR/Butyl-Lithium/Diisopropylamine/potassium tert-amylate, (iii) reducing the position 6 aldehyde with lithium aluminum hydride, (iv) deprotecting the protected regions of the estradiol compound. A reaction scheme for preparing estradiol derivatives is given below in Scheme 2.

The compounds of the present invention can be synthesized by the following methods as depicted in the schemes below.

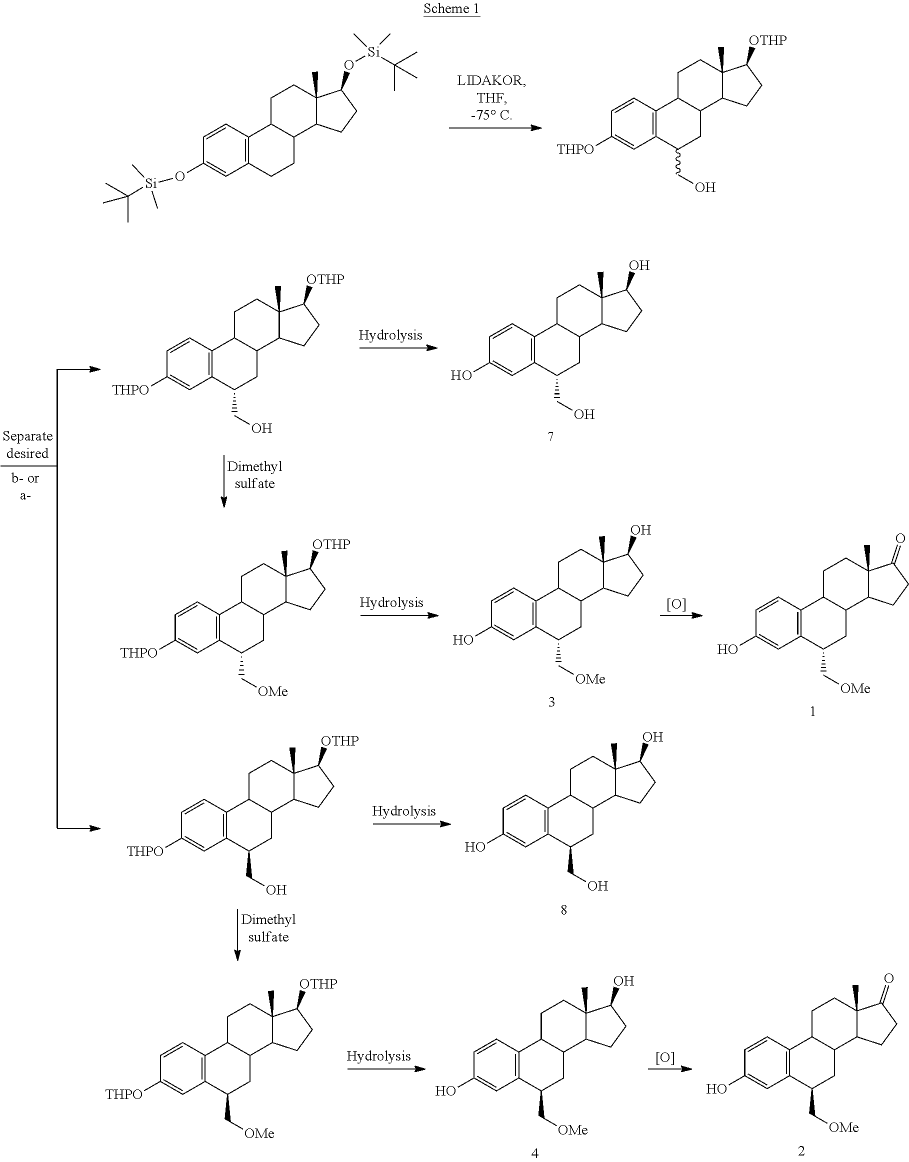

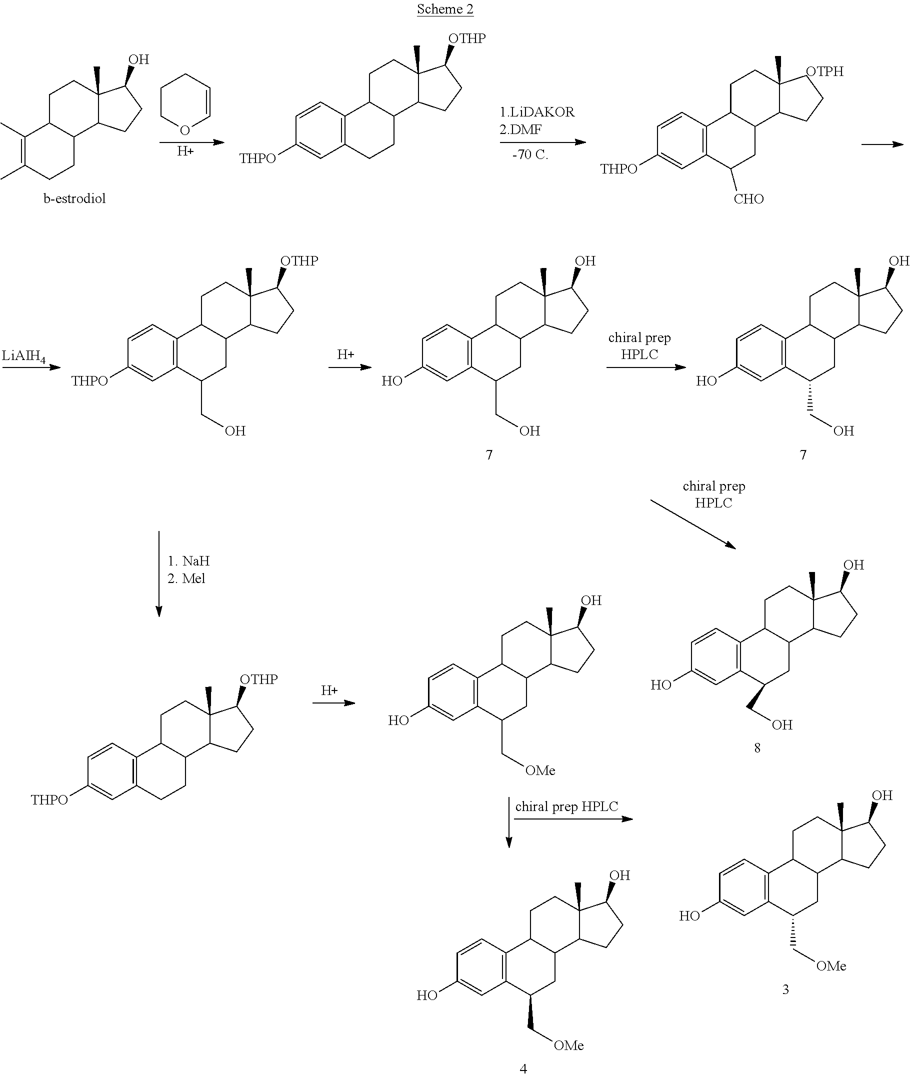

Various alkoxyalkyl derivatives, including compounds with C-6 alkoxyalkyl substituents, in accordance with this invention, involve selection of alkylating agents. Such derivatives would be understood by those skilled in art made aware of this invention, and is available through synthetic procedures of the sort described herein. Accordingly, without limitation, various C₁ to C₆ and higher alkyl and substituted alkyl reagents can be used as described herein to prepare the corresponding alkyloxyalkyl derivatives.

In another aspect of the invention, methods of making 6-amino derivatives of the estradiol are disclosed in reaction schemes below. Accordingly, 6-methoxylated estradiols described in Schemes 1-2 are employed and converted to their respective amino derivatives.

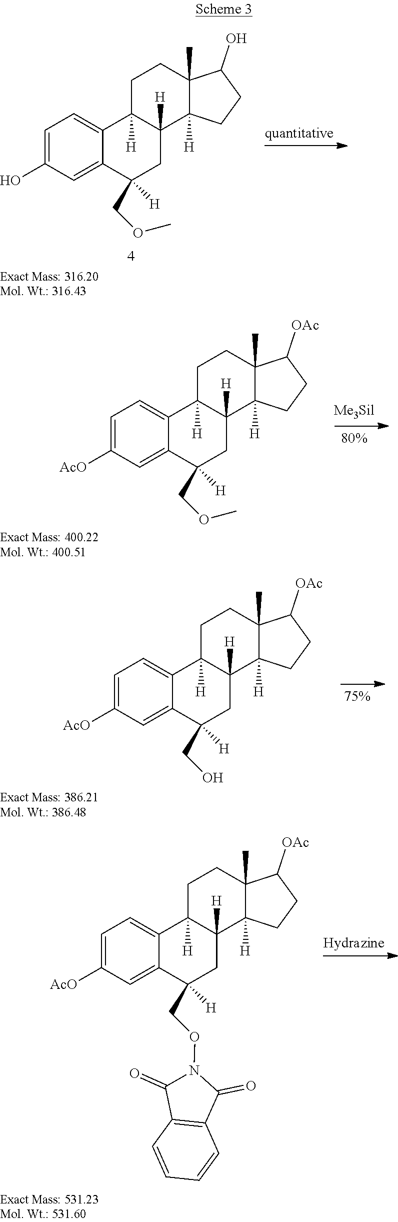

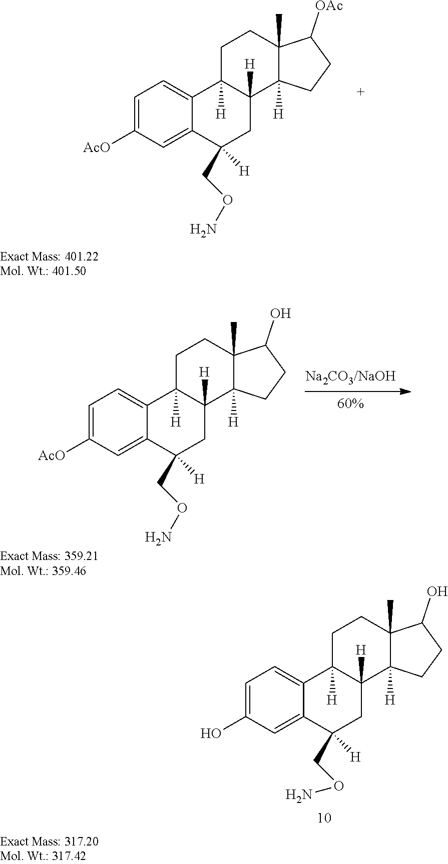

As noted herein, the salts of the compounds of this invention refer to non-toxic “pharmaceutically acceptable salts.” Other salts may, however, be useful in the preparation of the compounds according to the invention or of their pharmaceutically acceptable salts. When the compounds of the present invention contain a basic group, salts encompassed within the term “pharmaceutically acceptable salts” refer to non-toxic salts which are generally prepared by reacting the free base with a suitable organic or inorganic acid. Representative salts include any such salt known in the art. Where compounds of the present invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts.

As noted herein, the compounds of the present invention can be used in combination with other agents or other agents which will enhance the treatment regime for the mammalian subject. For example, the compounds of the methods could be used in combination with other active pharmaceutical ingredient (API). The individual components of such combinations can be administered separately at different times during the course of therapy or concurrently in divided or single combination forms to patients or regions of such patients in need of such therapy. The instant invention is therefore to be understood as embracing all such regimes of simultaneous or alternating treatment and the term “administering” is to be interpreted accordingly. It will be understood that the scope of combinations of the compounds of this invention with other agents useful to treat the targeted demyelinating disease includes in principle any combination with any pharmaceutical composition useful for treating disorders related to estrogen functioning.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following non-limiting examples and data illustrate various aspects and features relating to the compounds, compositions and/or methods of the present invention, including the synthesis of 6-substituted compounds, as are available through the methodologies described herein. In comparison with the prior art, the present compounds and methods provide results and data which are surprising, unexpected and contrary thereto. While the utility of this invention is illustrated through the preparation and use of several compounds, moieties and/or substituents thereof, it will be understood by those skilled in the art that comparable results are obtainable with various other compounds, moieties and/or substituents, as are commensurate with the scope of this invention.

### Example 1

The compounds of the methods are prepared according the procedures disclosed in U.S. Pat. Nos. 8,168,621 and 10,174,070, each of which is incorporated herein in its entirety. To exemplify the synthetic schemes described above and in detail in the '070 patent, the preparation of compound 21 (also referred to as compound NDC-1308 elsewhere herein) is provided in this Example 1.

### Methods for Preparing Compound 21 (Aka NDC-1308)

a) (8R,9S,13S,14S,17S)-3,17-bis(methoxymethoxy)-13-methyl-7,8,9,11,12,13,14,15,16,17-decahydro-6H-cyclopenta[a]phenanthrene—Chloromethyl methyl ether (7.0 mL, 92.0 mmol) is added to a solution of β-estradiol (5 g, 18.4 mmol) and diisopropylethylamine (16.0 mL 92 mmol) in 100 mL of THF. The reaction mixture is heated to reflux and stirred for 18 hours. The THF is removed in vacuo, and the yellow/brown oil is partitioned between water and CH₂Cl₂. The organic layer is separated, washed with brine, dried (Na₂SO₄), filtered, and evaporated in vacuo to give a golden oil. Purification by silica gel column chromatography (10% EtOAc/Hex) affords the title compound as a viscous, clear oil (5.7 g, 86%).

b) (8R,9S,13S,14S,17S)-3,17-bis(methoxymethoxy)-13-methyl-7,8,9,11,12,13,14,15,16,17-decahydro-6H-cyclopenta[a]phenanthren-6-ol—To a solution of potassium tert-butoxide (8.87 g, 79.0 mmol) and diisopropylamine (11.2 mL, 79.0 mmol) in 80 mL of anhydrous THF cooled to −78° C. under argon is added n-butyllithium (49.4 mL, 79.0 mmol, 1.6 M in hexane) dropwise. The reaction mixture is stirred at −78-° C. for 30-45 minutes. A solution of the compound from a) (5.7 g, 15.8 mmol) in 45 mL of THF is then added dropwise, and the reaction mixture is stirred for 3 hours at −78° C. During the addition of the compound from a), the reaction turns a deep red color. Trimethyl borate (10.6 mL, 94.8 mmol) is then added slowly, and the mixture is warmed to 0° C. and stirred for 2 hours. Hydrogen peroxide (24 mL of a 30% aq. solution) is then added, and the reaction mixture is warmed to room temperature and stirred for a further 1 hour. The reaction is cooled back to 0° C. and carefully quenched with a 10% aq. Na₂S2O₃ solution (70 ml). The resulting mixture is extracted with EtOAc (2×), and the combined organic extracts are dried (Na₂SO₄), filtered, and evaporated in vacuo to give a yellow/brown oil. Purification by silica gel column chromatography (25% EtOAc/Hex) affords the title compound as a white solid (3.5 g, 59%).

c) (8R,9S,13S,14S,17S)-3,17-bis(methoxymethoxy)-13-methyl-7,8,9,11,12,13,14,15,16,17-decahydro-6H-cyclopenta[a]phenanthren-6-one—Dess-Martin Periodinane (9.46 g, 22.3 mmol) is added portionwise to a solution of the compound from b) (7.0 g, 18.6 mmol) in 300 mL of CH₂C₀₂. The resulting reaction mixture stirred at room temperature for 3 hours. The mixture is poured into water and the layers are separated. The aqueous layer is extracted with CH₂Cl₂, and the combined organic extracts are washed with brine, dried (Na₂SO₄), filtered, and evaporated in vacuo to give a gooey, brown solid. Purification by silica gel column chromatography (15% EtOAc/Hex) affords the title compound as a pale yellow, viscous oil (6.0 g, 86%).

d) ethyl 2-(((8R,9S,13S,14S,17S)-3,17-bis(methoxymethoxy)-13-methyl-7,8,9,11,12,13,14,15,16,17-decahydro-6H-cyclopenta[a]phenanthren-6-ylidene)acetate—Triethyl phosphonoacetate (4.1 mL, 20.8 mmol) is added to a mixture of sodium hydride (832 mg, 20.8 mmol) in 25 mL of THF at room temperature. After approximately 10 minutes, a solution of the compound from c) (3.9 g, 10.4 mmol) in 10 mL of THF is added dropwise. The resulting reaction mixture is heated to reflux in a sealed tube for 72 hours. The mixture is concentrated in vacuo and purified by silica gel column chromatography (gradient from 5% EtOAc/Hex to 40% EtOAc/Hex) to give the title compound as a clear, viscous oil (3.4 g, 74%).

e) 2-((8R,9S,13S,14S,17S)-3,17-bis(methoxymethoxy)-13-methyl-7,8,9,11,12,13,14,15,16,17-decahydro-6H-cyclopenta[a]phenanthren-6-ylidene)ethanol—A solution of the compound from d) (3.1 g, 6.97 mmol) in 65 mL of THF is treated with lithium aluminum hydride (5.2 mL, 10.46 mmol, 2 M in THF) dropwise at 0° C. The cold bath is removed, and the reaction mixture is stirred at room temperature for 15 minutes. The reaction is cooled back to 0° C. and quenched by the careful addition of 1.3 mL of water, followed by 2.6 mL of 2N NaOH, and then 1.3 mL of water. The mixture is stirred vigorously until a white solid forms. The mixture is filtered, and the filtrate is concentrated in vacuo to give the title compound as a clear oil (2.8 g, 99%).

f) 2-((6S,8R,9S,13S,14S,17S)-3,17-bis(methoxymethoxy)-13-methyl-7,8,9,11,12,13,14,15,16,17-decahydro-6H-cyclopenta[a]phenanthren-6-yl)acetaldehyde—A mixture of the compound from e) (3.09 g, 7.68 mmol) and 10% Pd/C (500 mg) in 100 mL of ethyl acetate is stirred under 40 psi of H₂ (g) for 5 hours at room temperature. The mixture is filtered through Celite, and the Celite is washed well with ethyl acetate. The filtrate is concentrated in vacuo to give a pale yellow oil (3.1 g). The oil is dissolved in 100 mL of dichloromethane, and Dess-Martin Periodinane (3.9 g, 9.22 mmol) is added portionwise. The resulting reaction mixture is stirred at room temperature for 30 minutes. The mixture is poured into water and extracted with CH₂Cl₂. The combined organic extracts are washed with brine, dried (Na₂SO₄), filtered, and evaporated in vacuo to give a brown solid. Purification by silica gel column chromatography (15% EtOAc/Hex) affords the title compound as a clear oil (2.0 g, 65%).

g) 4-((6R,8R,9S,13S,14S,17S)-3,17-bis(methoxymethoxy)-13-methyl-7,8,9,11,12,13,14,15,16,17-decahydro-6H-cyclopenta[a]phenanthren-6-yl)but-2-en-1-ol—Lithium bis(trimethylsilyl)amide (18.4 mL, 18.4 mmol, 1.0 M in THF) is added dropwise to a suspension of (2-hydroxyethyl) triphenylphosphonium bromide (3.37 g, 8.70 mmol) in 60 mL of THF at 0° C. After 1 hour, the golden brown solution is treated with a solution of the compound from f) (1.4 g, 3.48 mmol) in 10 mL of THF dropwise. The resulting reaction mixture is stirred at 0° C. for 40 minutes and then quenched with saturated aqueous NH₄Cl. The resulting mixture is extracted with EtOAc (2×), and the combined organic extracts are dried (Na₂SO₄), filtered, and evaporated to give a brown oil. Purification by silica gel column chromatography (gradient from 20% EtOAc/Hex to 75% EtOAc/Hex) affords the title compound as a yellow, viscous oil (680 mg, 45%).

h) 4-((6R,8R,9S,13S,14S,17S)-3,17-bis(methoxymethoxy)-13-methyl-7,8,9,11,12,13,14,15,16,17-decahydro-6H-cyclopenta[a]phenanthren-6-yl)but-2-enal—Dess-Martin Periodinane (437 mg, 1.03 mmol) is added to a solution of the compound from g) (370 mg, 0.86 mmol) in 15 mL of CH₂Cl₂ at room temperature. The resulting reaction mixture is stirred for 10 minutes and then poured into water. The layers are separated and the aqueous layer is extracted with CH₂Cl₂ (2×). The combined organic extracts are washed with brine, dried (Na₂SO₄), filtered, and evaporated in vacuo to give a brown oil. Purification by silica gel column chromatography (gradient from 5% EtOAc/CH₂Cl₂ to 10% EtOAc/CH₂Cl₂) affords the title compound as a pale yellow, viscous oil (358 mg, 86%).

i) 6-((6R,8R,9S,13S,14S,17S)-3,17-bis(methoxymethoxy)-13-methyl-7,8,9,11,12,13,14,15,16,17-decahydro-6H-cyclopenta[a]phenanthren-6-yl)hexa-2,4-dien-1-ol—Lithium bis(trimethylsilyl)amide (4.3 mL, 4.29 mmol, 1.0 M in THF) is added dropwise to a suspension of (2-hydroxyethyl) triphenylphosphonium bromide (786 mg, 2.03 mmol) in 14 mL of THF at 0° C. After 30 minutes, the golden brown solution is treated with a solution of the compound from h) (345 mg, 0.81 mmol) in 2 mL of THF dropwise. The resulting reaction mixture is stirred at 0° C. for 20 minutes and quenched with saturated aqueous NH₄Cl. The resulting mixture is extracted with EtOAc (2×), and the combined organic extracts are dried (Na₂SO₄), filtered, and evaporated to give a brown oil. Purification by silica gel column chromatography (gradient from 5% EtOAc/CH₂Cl₂ to 40% EtOAc/CH₂Cl₂) affords the title compound as a yellow, viscous oil (140 mg, 38%).

j) (6R,8R,9S,13S,14S,17S)-6-(6-methoxyhexa-2,4-dien-1-yl)-3,17-bis(methoxymethoxy)-13-methyl-7,8,9,11,12,13,14,15,16,17-decahydro-6H-cyclopenta[a]phenanthrene—A solution of the compound in i) (135 mg, 0.3 mmol) is cooled to 0° C., and sodium hydride (120 mg, 3.0 mmol) is added portionwise. After 5-10 minutes, iodomethane (0.19 mL, 3.0 mmol) is added dropwise, and the resulting reaction mixture is warmed to room temperature and stirred for 4 hours. EtOAc is added and the reaction is carefully quenched with water. The layers are separated and the organic layer is dried (Na₂SO₄), filtered, and evaporated to give a brown oily residue. Purification by silica gel column chromatography (gradient from 5% EtOAc/Hex to 20% EtOAc/Hex) affords the title compound as a clear oil (92 mg, 65%).

k) (6R,8R,9S,13S,14S,17S)-6-(6-methoxyhexyl)-3,17-bis(methoxymethoxy)-13-methyl-7,8,9,11,12,13,14,15,16,17-decahydro-6H-cyclopenta[a]phenanthrene—A mixture of the compound in j) (90 mg, 0.19 mmol) and 10% Pd/C (100 mg) in 5-10 mL of ethyl acetate is stirred under a balloon of H₂ (g) for 16 hours at room temperature. The mixture is filtered through Celite, and the Celite is washed well with ethyl acetate. The filtrate is concentrated in vacuo to give the title compound as a clear oil (90 mg, 99%).

l) (6R,8R,9S,13S,14S)-6-(6-methoxyhexyl)-13-methyl-7,8,9,11,12,13,14,15,16,17-decahydro-6H-cyclopenta[a]phenanthrene-3,17-diol (Compound 21)—A solution of the compound from k) (90 mg, 0.19 mmol) in 1.5 mL each of 6 N HCl and THF is stirred for 5 hours at room temperature. The reaction mixture is diluted with water and extracted with EtOAc (2×). The combined organic extracts are dried (Na₂SO₄), filtered, and evaporated in vacuo to give a clear, oily residue. Purification by silica gel column chromatography (gradient from CH₂Cl₂ to 30% EtOAc/CH₂Cl₂) afforded Compound 21 (NDC-1308) as a white solid foam (38 mg, 52%).

### Example 2

### Methods for Preparing Compounds 3 and 4

As outlined in Scheme 2, estradiol derivatives compounds 3 and 4 are synthesized in the following manner. The protected estradiol compound is prepared by reaction of β-estradiol with dihydropyran in THF, using toluenesulfonic acid or camphorsulfonic acid as catalyst. As one of ordinary skill in the art can appreciate, this reaction is an equilibrium reaction and does not go to completion under such conditions. Thus, both the mono-protected estradiols can be found in the reaction mixture. Such crude reaction mixture undergoes a trituration step with acetonitrile causing the desired bis-TIP estradiol to crystallize in approximately 70% yield.

As shown in Scheme 2, the key intermediate is obtained via acylation at the benzylic 6-position with the strong base mixture referred to as LiDAKOR: butyl lithium, diisopropylamine, and potassium tert-amylate. Under such conditions at −70° C., one of ordinary skill in the art can appreciate the abstraction of a proton at a benzylic position. The intermediate is then purified by column chromatography to give a syrup in approximately 50% yield, still containing minor impurities and column solvents. Reduction of the aldehyde with an excess of lithium aluminum hydride results in high yields of the racemic hydroxymethyl estradiol compound.

For purposes of preparing compounds 3 and 4, the methoxymethyl compound is prepared by methylation of hydroxymethyl estradiol compound with sodium hydride and methyl iodide. The methoxymethyl compound is purified by column chromatography to give a glassy foam. Deprotecting the protected groups give racemic 6-methoxymethyl estradiol compound. Separation of the enantiomers is performed using chiral preparative HPLC to give the compounds 3 and 4. For compound 4, a chiral purity of >95:5 R:S is realized. For compound 3, a chiral purity of 86:14 S:R is realized. It is well within the level of one of ordinary skill in the art to employ NMR for determination of the absolute stereochemistry of the 6-position, where the 4- and 6-protons are diagnostic.

### Example 3

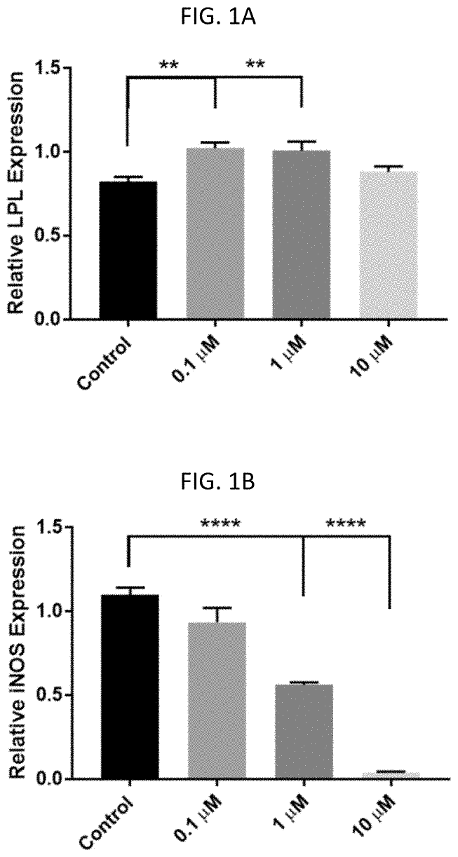

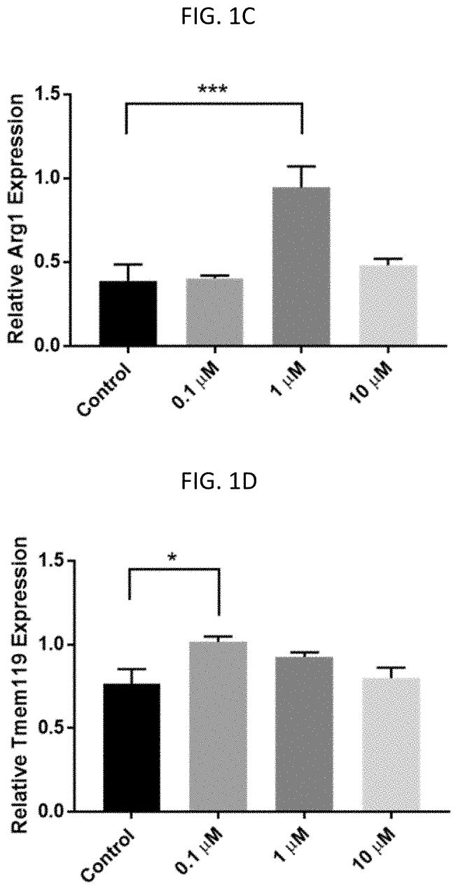

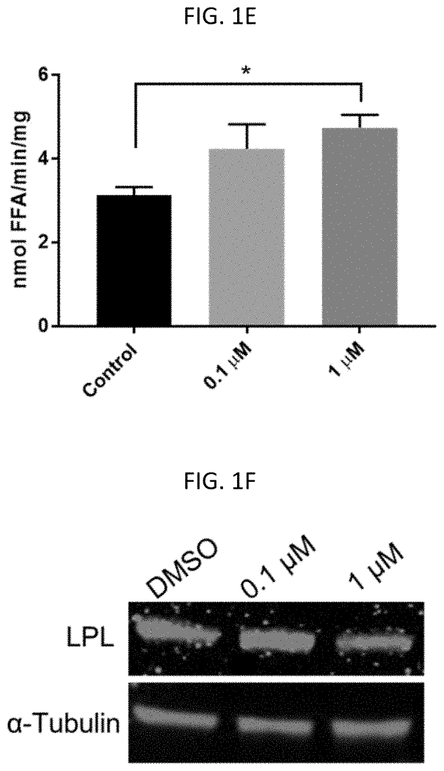

Experiments were conducted with primary (neonatal) rat microglia. The experimental time scale was amended to account for extended culture time. It was found that following 72 hours of NDC-1308 (compound 21) supplementation, LPL expression and hydrolytic activity (to produce free fatty acid, FFA) was increased (FIGS. 1A, 1E, and 1F). Moreover, the expression of inflammatory genes (e.g. iNOS) were reduced after NDC-1308 supplementation (FIG. 1i), whereas the expression of reparative genes (e.g. Arg1) were increased with a similar supplementation regimen (FIG. 1C). As would be understood by those skilled in the art and made aware of this invention, such results support promotion of the microglial anti-inflammatory (M2) phenotype from the pro-inflammatory (M1) phenotype.

### Example 4

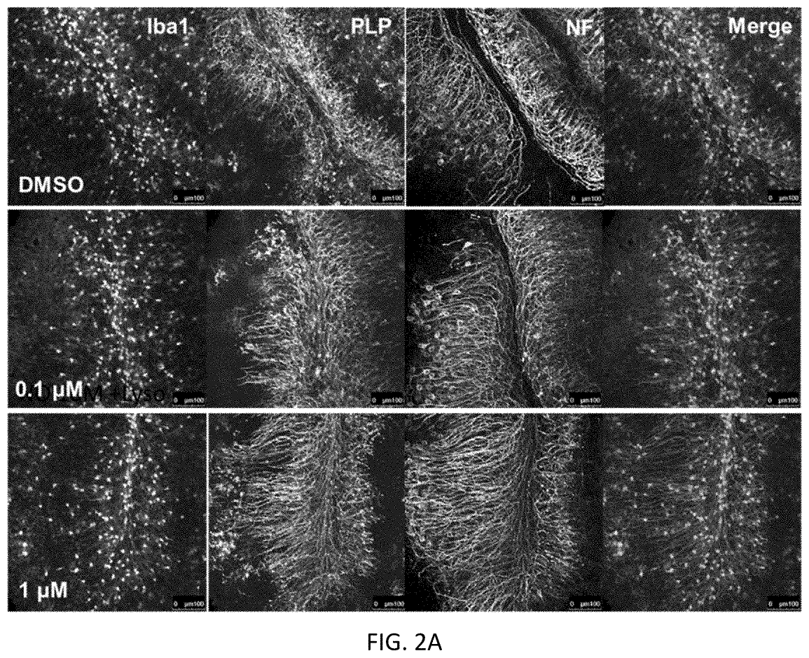

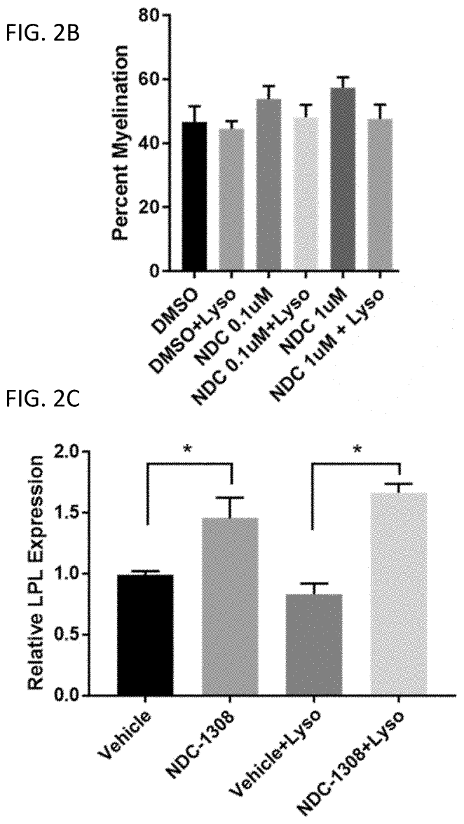

Based on these findings, additional experiments were designed to determine whether compounds of the sort described herein show up-regulation on LPL expression and activity in primary microglial cells, and, if so, whether this change in phenotype and increase in LPL activity translates to an increase in microglial phagocytosis and clearance. With reference to FIGS. 2A-2C, organotrophic cerebellar cultures were pre-treated with NDC-1308 and demyelinated with lysolecithin. Brain slices were then allowed to recover and remyelinate for up to 7 days. At 7 days post lysolecithin markers of microglia and myelination were analyzed. Data suggest that supplementation with NDC-1308 improves remyelination outcomes (FIG. 2A-2B). In addition, LPL expression was measured 3 days post-lysolecithin treatment, since LPL expression is highest at the transition between de- and re-myelination. It was found that NDC-1308 supplementation increased LPL expression in both lysolecithin-treated and control cerebellar cultures (FIG. 2C).

In accordance with the foregoing, NDC-1308 shows consistent up-regulation of LPL expression and activity in primary microglial cells (as supported by improved remyelination). With increasing concentration, NDC-1308 appears to down regulate the expression of pro-inflammatory genes (e.g., Arg1), consistent with polarization to an M2-like phenotype.

### Example 5

Three human tumor cell lines, A-549, Panc-1, and SK-OV-3, were each grown in two flasks cultured to roughly 50% confluence. Cells were treated for 24 hours with NDC-1308 or 10% DMSO at concentrations of compound specified in Table 1. At termination, the cells were scraped free and washed in ice-cold PBS, collected by centrifugation, and immediately frozen at −80° C.

Total RNA was prepared from the frozen tissue samples using Trizol-based cell lysis followed by 65° C. hot phenol extraction and RNeasy chromatography purification (Qiagen, Valencia, Calif.). The concentration of RNA was determined by measuring the absorbance at 260 nm (A260). RNA purity was assessed by confirming that extracted RNAs had an A260/A280 ratio of 1.8 or greater. The RNA was then tested for relative integrity by determining the ratio of intact 28S and 18S ribosomal RNAs, by capillary electrophoresis with the Agilent 2100 BioAnalyzer (Agilent Technologies, Santa Clara, Calif.). All RNAs accepted for array analysis had ratios exceeding 1.0.

RNAs were labeled using 1 μg of RNA as input to Low Input labeling reaction (Agilent Technologies) with Cy5 (650 nm emitter) and reference RNA was labeled with Cy3 (550 nm emitter) nucleotides. Labeling, hybridizations and subsequent washings were carried out on with the Human GE 4x44K v2 G4110B microarray kits from Agilent Technologies, according to the manufacturer's instructions. The resulting hybridized chips were scanned (G2505 Scanner, Agilent Technologies), and intensity information for each detector spot was extracted from the scanned image using Agilent feature extraction software version 10.5.1.1. A threshold of P≤0.001 was used as the cutoff point for significant change in mRNA abundance between the compound treated and vehicle treated samples.

Incubation of NDC-1308 with three human cell lines showed on average approximately 5-fold up-regulation of the LPL gene (Log 2) and an approximately 7-fold up-regulation of the ApoC2 gene (Log 2). Transcriptional modulation was specific to LPL and ApoC2. Because the addition of LPL protein to microglial cultures causes the polarization of macrophages to the M2-like state (anti-inflammatory, pro-repair), the data is consistent with a polarization to an M2-like phenotype.

TABLE 1
Gene Expression

| | | | Gene Expression Values | | |
|---|---|---|---|---|---|
| | | | (fold increase-log2) | | |
| | | Entrez | A549 | SKOV3 | Panc1 |
| | | Gene | (lung) | (ovary) | (pancreas) |
| Gene Name | Symbol | Gene ID | 10 μm | 10 μm | 10 μm |
| Lipoprotein Lipase | LPL | 4023 | 5.18 | 5.17 | 5.38 |
| Apolipoprotein C2 | ApoC2 | 344 | 9.00 | 6.55 | 7.27 |
| Apolipoprotein C3 | ApoC3 | 345 | 0 | 0 | 0 |
| Apolipoprotein E | ApoE | 348 | 0 | 0 | 0 |
| Apolipoprotein A1 | ApoA1 | 335 | 0 | 0 | 0 |

p <0.001

1. A compound of formula 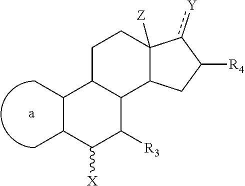
wherein the “a” ring is selected from the group consisting of 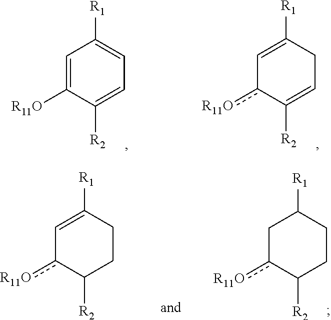
R₁, R₂, R₃ and R₄ are independently selected from hydrogen, C₁-C₆ alkyl, halo, a sulfate, a glucuronide, —OH, a bulky group, aryl, cycloalkyl, heteroaryl, heterocycloalkyl, —NH(CH₂)ₙCH₃; a phosphate group, and a phosphinate group;
R₁₁ is selected from H, C₁-C₆ alkyl, halogen, a sulfate, a glucoronide, —SO₂NH₂, —COOH, —CN, —CH₂CN—, —NHCN—, —CHO, ═CHOCH₃, —COO salt, —OSO₂alkyl, —NH₂, and —NHCO(CH₂)ₙ;
X is selected from C₁-C₁₂ alkyl, C₂-C₁₂ alkenyl, C₂-C₁₂ alkynyl, halogen, a glucoronide, —NH₂, —SO₂NH₂, —COOH, —CN, —CH₂CN, —NHCN, —CHO, —COOsalt, —OSO₂alkyl, —SH, —CH₃, —CH[(CH₂)ₙCH₃]COOCH₃, —(CH₂)ₘCOOCH₃, —(CH₂)ₘ—O—CH₃, —(CH₂)ₘ—O—(CH₂)ₙCH₃, (CH₂)ₘ—S—CH₃, —(CH₂)ₘ—S—(CH₂)ₙCH₃, —(CH₂)ₘ—NH—(CH₂)ₙCH₃, —C₂-C₈ alkenyl-O—(CH₂)ₙCH₃, —C₂-C₈ alkenyl-S—(CH₂)ₙCH₃, —C₂-C₈ alkenyl-NH—(CH₂)ₙCH₃, —C₂-C₈ alkynyl-O—(CH₂)ₙCH₃, —C₂-C₈ alkynyl-S—(CH₂)ₙCH₃, —C₂-C₈ alkynyl-NH—(CH₂)ₙCH₃, —(CH₂)ₘ—OH, —(CH₂)ₘ—NH₂, —(CH₂)ₘ—O—NH₂, —(CH₂)ₘ—S—NH₂, —NH(CH₂)ₘCH₃, —NH(CH₂)ₘOCH₃, —NH(CH₂)ₘCHOH—COOH, —N(CH₃)₂, —(CH₂)ₘ(NH)CH₂OH, —NHCOOH, —(CH₂)ₘNHCOOH, —NO₂, —SCN, —SO₂alkyl, —B(OH)₂, —(CH₂)ₘN(CH₃)—SO₂—NH₃, —(CH₂)ₘ—NH—SO₂—NH₂, —NHC(═S)CH₃, and —NHNH₂;
Y is selected from hydrogen, ═O, —OCO(C₁-C₂₀ alkyl) and —OH; and
Z is H or methyl;
wherein m is an integer between 0-20,
n is an integer between 0-8,
the [symbol] symbol represents either a single or a double bond capable of forming a keto group at position 3 and/or 17;
the [symbol] symbol represents any type of bond regardless of the stereochemistry; and
the respective enantiomers, other stereochemical isomers, hydrates, solvates, tautomers and pharmaceutically acceptable salts of said compounds.

2. A method for modulating lipoprotein lipase (LPL) or apolipoprotein C₂ (ApoC2) expression and/or activity, the method comprising contacting a cell capable of expressing LPL and/or ApoC2 with an effective amount of the compound according to claim 1.

3. The method of claim 2, wherein the cell is a microglial cell or a macrophage cell.

4. The method of claim 2, wherein LPL and/or ApoC2 is upregulated.

5. A method for switching, polarizing, or altering an inflammatory phenotype, the method comprising contacting a cell having a pro-inflammatory phenotype with an effective amount of the compound according to claim 1, thereby switching, polarizing, or altering the inflammatory phenotype to an anti-inflammatory phenotype.

6. The method of claim 5, wherein the cell is a microglial cell or a macrophage cell.

7. The method of claim 5, wherein an inflammatory gene is down-regulated.

8. The method of claim 5, wherein a reparative gene is upregulated.

9. A method for treating a condition, disease, or disorder in a subject, the method comprising administering a therapeutically-effective amount of the compound according to claim 1 to the subject, wherein the subject is in need of a treatment for inflammation.

10. The method of claim 9, wherein the subject is in need of a treatment for inflammation associated with a cell having a pro-inflammatory phenotype and wherein administration of the therapeutically-effective amount of the compound switches, polarizes, or alters the pro-inflammatory phenotype to an anti-inflammatory phenotype.

11. The method of claim 10, wherein the inflammation is induced by an infection.

12. The method of claim 11, wherein the inflammation is induced by a pulmonary viral infection.

13. The method of claim 10, wherein the compound is administered via an inhaler, a nebulizer, or a nasal spray device.

14. The method of claim 10, wherein the cell is a microglial cell or a macrophage cell.

15. A method for treating a condition, disease, or disorder in a subject, the method comprising administering a therapeutically-effective amount of the compound according to claim 1 to the subject, wherein the subject is in need of a treatment for a peripheral tissue disease state.

16. The method of claim 15, wherein the peripheral tissue disease state is type-2 diabetes, cardiovascular disease, or obesity.

17. A method for treating a condition, disease, or disorder in a subject, the method comprising administering an effective amount of the compound according to claim 1 to the subject, wherein the subject is in need of a treatment for a central nervous system tissue disease state associated with a cell having a pro-inflammatory phenotype and wherein administration of the effective amount of the compound switches, polarizes, or alters the pro-inflammatory phenotype to an anti-inflammatory phenotype.

18. The method of claim 17, wherein the cell is a microglial cell or a macrophage cell.

19. The compound of claim 1, wherein the compound is of formula 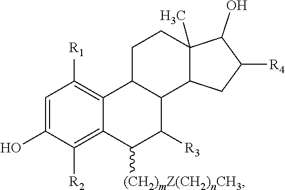
wherein Z is selected from —O—, —S— and —NH—,
wherein m is 1-12
wherein n is 0-4,
wherein R₁ is selected from hydrogen, —OH and halo,
wherein R₂ is selected from hydrogen and halo,
wherein R₃ is selected from hydrogen, —OH and halo, and
wherein R₄ is selected from hydrogen, halo and C₁-C₆ alkyl.

20. The compound of claim 19, wherein the compound is [chemical structure]
